(19) United States
(12) Patent Application Publication
(10) Pub. No.: US 2012/0264805 A1
(43) Pub. Date: Oct. 18, 2012

(54) MEDICAMENT FOR THE TREATMENT AND PREVENTION OF LIVER FAILURE

(75) Inventors: Amar Sharma, Hannover (DE); Tobias Cantz, Hannover (DE); Michael Ott, Hannover (DE); Nidhi Narain, New Delhi (IN)

(73) Assignee: MEDIZINISCHE HOCHSCHULE HANNOVER, HANNOVER (DE)

(21) Appl. No.: 13/088,086

(22) Filed: Apr. 15, 2011

(51) Int. Cl. A61K 31/713; C12N 15/63; A61P 1/16; A61K 31/7105

(52) U.S. Cl. 514/44 A; 435/320.1

(57) ABSTRACT

Small inhibitory RNA (siRNA) molecules, which e.g. are purified and/or isolated, as an active in a medicament, which siRNA molecules are inhibitory RNA molecules that through RNA interference (RNAi) reduce or prevent expression of the p53 upregulated modulator of apoptosis (PUMA). The siRNA molecules can be administered as a medicament to a patient suffering from an impaired liver function or from liver damage for the treatment of a functionally impaired liver, for delaying a deterioration of liver function and/or for prevention of liver failure, especially in patients who suffer from a critical impairment of damage to the liver, e.g. for delaying the complete failure of liver function.

### FIELD

A field of the invention is liver treatment. The present invention relates to a medicament for the treatment or prevention of liver failure, especially of acute liver failure, including subfulminant and fulminant liver failure. Generally, liver failure can be induced by viral agents, e.g. an infection by hepatitis B virus and/or by drug abuse, including abuse of alcohol and acetaminophen (paracetamol).

### REFERENCE TO SEQUENCE LISTING

This application incorporates by reference the sequence listing in the electronic sequence listing filed herewith and as an appendix to this application.

### BACKGROUND

Lee in the New England Journal of Medicine, 1862-1872 (1993) describes causes for acute liver failure. For the treatment of acute liver failure, a treatment directed against the causative agent is currently used, e.g. an antiviral therapy in the case of viral infection, or the administration of a chemical antidote in the case of drug abuse, e.g. acetylcystein in the case of paracetamol intoxication. In severe cases, liver transplantation if a suitable donor organ is available, is considered the therapy of choice.

Generally, it is known that microRNAs (miRNAs) are non-coding small RNA molecules, e.g. 21 or 23 nucleotides in length, which naturally regulate gene expression by interfering with mRNA molecules.

### SUMMARY OF THE INVENTION

The invention provides a medicament for the treatment of a functionally impaired liver, for delaying a deterioration of liver function and/or for prevention of liver failure, containing in a pharmaceutically acceptable formulation an siRNA or microRNA having a nucleic acid sequence which hybridizes to the mRNA encoding. PUMA. In a preferred embodiment, the siRNA or microRNA is reverse complementary to 3′ UTR of the mRNA encoding PUMA, which 3′UTR has the sequence of SEQ ID NO: 1. In a preferred embodiment, the siRNA or microRNA comprises a sequence selected from the group consisting of nucleotide sequences having at least 85% sequence similarity to one of SEQ ID NO 2 to SEQ ID NO 84.

The invention provides a purified nucleic acid construct containing a sequence encoding an siRNA having a nucleic acid sequence which hybridizes to the mRNA encoding PUMA as a medicament for the treatment of a functionally impaired liver, for delaying a deterioration of liver function and/or for prevention of liver failure.

The invention provides a method for treating, delaying and/or preventing liver failure in humans or animals, the method comprising the step of providing a nucleic acid having which hybridizes to the mRNA encoding PUMA in a pharmaceutical formulation suitable for administration to the human or animal

In preferred embodiments, the siRNA or microRNA is reverse complementary to 3′ UTR of the mRNA encoding PUMA, which 3′UTR has the sequence of SEQ ID NO: 1. In preferred embodiments, the siRNA or microRNA comprises a sequence selected from the group consisting of nucleotide sequences having at least 85% sequence similarity to one of SEQ ID NO 2 to SEQ ID NO 84.

### BRIEF DESCRIPTION OF THE DRAWINGS

FIG. 1 shows a table of the relative cell viability of hepatocytes after induction of apoptosis in cells transfected with a nucleic acid construct for expression of the microRNAs (miRNA mimic) as indicated in relation to cell viability for expression of a non-specific control microRNA (control mimic),

FIG. 2 shows a graph of the relative cell viability of hepatocytes in the WST assay for over-expression of miR-221 (miR-221 mimic) and for over-expression of an inhibitor of miR-221 (miR-221-inhibitor) in relation to viability of mock-transfected control cells (control, set to 1), at 12 hours and 24 hours, respectively, following induction of apoptosis,

FIG. 3 shows a graph of the relative cell viability of hepatocytes in the WST assay for over-expression of miR-709 (miR-709 mimic) and for over-expression of an inhibitor of miR-709 (miR-709-inhibitor) in relation to viability of mock-transfected control cells (control, set to 1), at 12 hours and 24 hours, respectively, following induction of apoptosis,

FIG. 4 shows the result of the assay for caspase-3/7 activity in hepatocytes for over-expression of miR-221 (miR-221 mimic) and for over-expression of an inhibitor of miR-221 (miR-221-inhibitor) in relation to control cells (100%),

FIG. 5 shows the result of the assay for caspase-3/7 activity in hepatocytes for over-expression of miR-709 (miR-709 mimic) and for over-expression of an inhibitor of miR-709 (miR-709-inhibitor) in relation to control cells (100%),

FIG. 6 shows FACS results of hepatocytes transfected with a control microRNA (miRNA) and of cells transfected with a nucleic acid construct expressing miR-221 after induction of apoptosis,

FIG. 7 shows the result of the assay for caspase-3/7 activity in shDGCR8 hepatocytes transfected with miR-221 in comparison to mock or non-specific (control),

FIG. 8 schematically shows integrating nucleic acid constructs for permanent expression of Cre recombinase (Cre) as a non-specific control, and for miR-221,

FIG. 9 shows micrographs of liver sections of ROSA26 reporter mice transduced in vivo with 1×10¹¹ viral particles encoding Cre in X-gal staining (top), of HE stained liver sections of Balb/C mice transduced in vivo with viral particles encoding Cre as a control (middle), and HE stained liver sections of Balb/C mice transduced in vivo with viral particles encoding miR-221 in an expression cassette for permanent transfection (lower),

FIG. 10 shows quantitative RT-PCR results for over-expression of miR-221 in livers of mice transduced with viral particles containing a nucleic acid construct with an expression cassette for miR-221,

FIG. 11 shows survival curves (Kaplan-Meier) of mice having received a nucleic acid construct with an expression cassette for miR-221 and Cre (non-specific control) by administration of a viral vector after induction of lethal liver failure,

FIG. 12 shows the result of quantitative RT-PCR from liver tissue of mice that had received the viral vector containing a nucleic acid construct with an expression cassette for miR-221 and Cre (non-specific control),

FIG. 13 shows results of the caspase-3/7 activity assay in mice transduced with a viral vector containing a nucleic acid construct with an expression cassette for miR-221 or Cre (non-specific control), at 9 h after induction of apoptosis,

FIG. 14 shows micrographs of liver sections of the mice transduced with a viral vector containing a nucleic acid construct with an expression cassette for miR-221 or Cre (non-specific control) in HE and TUNEL staining,

FIG. 15 shows a Western blot for PUMA and tubulin from Balb/C mouse livers at Oh and 12 h after induction of apoptosis by Jo2 injection, positive control for PUMA was NIH3T3 lysate,

FIG. 16 shows mRNA levels for PUMA in mouse hepatocytes at 48 h after transfection with a nucleic acid construct expressing miR-221 at 0 h and 12 h after induction of apoptosis,

FIG. 17 shows relative luciferase activities in primary hepatocytes expressing miR-Glo-PUMA reporter for co-transfectants transcribing the wt 3′UTR with non-specific Cre (control miRNA mimic), the wt 3′UTR with miR-221 (miR-221 mimic), and with a mutated 3″UTR,

FIG. 18 shows relative luciferase activities in primary hepatocytes expressing miR-Glo-PUMA reporter for co-transfectants transcribing the wt 3′UTR with non-specific Cre (control miRNA mimic), the wt 3′UTR with miR-221 inhibitor (miR-221inhibitor), and a mutated 3′UTR with the miR-221 inhibitor, showing that miR-221 binding to the wt UTR of PUMA inhibits reporter expression, which represents PUMA,

FIG. 19 shows a Western blot for PUMA and tubulin in hepatocytes at 48 h after transfection with non-specific control (control miRNA mimic) or with miR-221 (miR-221 mimic) showing decrease in expression of PUMA in presence of miR-221,

FIG. 20 shows a Western blot for PUMA and tubulin in hepatocytes at 48 h after transfection with non-specific control (control miRNA mimic) or with miR-221 inhibitor (miR-221 inhibitor) showing increase in expression of PUMA in presence of miR-221 inhibitor,

FIG. 21 shows measurement results in the WST assay in hepatocytes transfected with a first or a second siRNA specific for PUMA mRNA (puma siRNA-1 and puma siRNA-2, resp.) after induction of apoptosis, showing increased viability compared to mock and unspecific control transfected cells,

FIG. 22 shows measurement results in the caspase-3/7 assay in hepatocytes transfected with a first or a second siRNA specific for PUMA mRNA (puma siRNA-1 and puma siRNA-2, resp.) after induction of apoptosis, showing lower apoptosis compared to mock and unspecific control transfected cells,

FIG. 23 shows Western blot analyses on liver tissue lysates from mice transduced with a viral vector containing a nucleic acid construct with a constitutive (ttr) expression cassette for miR-221 or Cre (non-specific control) showing reduced translation of PUMA, PTEN, and p27 in presence of miR-221 only,

FIG. 24 shows results for the WST assay on hepatocytes transfected with siRNA specific for Puma, for Pten or for Bmf, respectively, and transfected by a combination of one siRNA specific for Puma, for Pten or for Bmf each with miR-221, showing an increase in cell viability for hepatocytes containing miR-221, and

FIG. 25 shows results for the caspase-3/7 assay on hepatocytes transfected with siRNA specific for Puma, for Pten or for Bmf, respectively, and transfected by a combination of one siRNA specific for Puma, for Pten or for Bmf each with miR-221, showing a decrease in apoptosis for hepatocytes containing miR-221,

Generally, in the figures * indicates P<0.05, and ** indicates P<0.005.

### DETAILED DESCRIPTION OF THE PREFERRED EMBODIMENTS

The invention provides small inhibitory RNA (siRNA) molecules, which e.g. are purified and/or isolated, as an active in a medicament, which siRNA molecules are inhibitory RNA molecules that through RNA interference (RNAi) reduce or prevent expression of the p53 upregulated modulator of apoptosis (PUMA). The siRNA molecules can be administered as a medicament to a patient suffering from an impaired liver function or from liver damage for the treatment of a functionally impaired liver, for delaying a deterioration of liver function and/or for prevention of liver failure, especially in patients who suffer from a critical impairment of damage to the liver, e.g. for delaying the complete failure of liver function.

The siRNA molecules (siRNAs) can comprise or consist of single-stranded or double-stranded oligonucleotides of RNA and/or DNA hybridizing to the mRNA encoding PUMA, including siRNA molecules hybridizing under physiological conditions to the mRNA encoding PUMA, which siRNA molecules are at least 85%, preferably at least 90%, more preferably at least 95% reverse complementary of a section of at least 19 to at least 23 nucleotides length, preferably hybridizing to the 3′UTR (3′-untranslated region) of the mRNA (SEQ ID NO: 1) encoding PUMA, and can be in the form of short-hairpinRNA (shRNA) or comprised in a microRNA or in a microRNA precursor. In one embodiment, the siRNA molecule is the active in the medicament, e.g. formulated as liposomes, and in an alternative embodiment, the siRNA is encoded in an expression cassette of a nucleic acid construct which is formulated as a medicament, e.g. as a viral particle or as a virus-like particle. The siRNA, or combination of two or more of these, is preferably contained in a microRNA molecule or encoded in an expression cassette of a nucleic acid construct as a microRNA. Exemplary microRNAs are given as SEQ ID NO 2 to SEQ ID NO 84, of which the group comprising or consisting of miR-221 (SEQ ID NO 2), miR-222 (SEQ ID NO 3), miR-709 (SEQ ID NO 4), miR-1827 (SEQ ID NO 5), miR-148 (SEQ ID NO 6), miR-152 (SEQ ID NO 7), miR-144 (SEQ ID NO 8), miR-145 (SEQ ID NO 9), miR-139-5p (SEQ ID NO 10), miR-27a (SEQ ID NO 11), miR-27b (SEQ ID NO 78), miR-34a (SEQ ID NO 12), miR-34b-5p (SEQ ID NO 13), miR-34c (SEQ ID NO 14), miR-34c-5p (SEQ ID NO 15), miR-449 (SEQ ID NO 84), miR-449a (SEQ ID NO 16) miR-449b (SEQ ID NO 17), miR-449c (SEQ ID NO 79), miR-699 (SEQ ID NO 18), miR-24 (SEQ ID NO 19), miR-143 (SEQ ID NO 20), miR-101 (SEQ ID NO 21), miR-150 (SEQ ID NO 22), miR-128 (SEQ ID NO 23), miR-125 (SEQ ID NO 24), miR-351 (SEQ ID NO 25), miR-140 (SEQ ID NO 26), miR-140-5p (SEQ ID NO 27), miR-876-3p (SEQ ID NO 28), miR-205 (SEQ ID NO 29), miR-29a (SEQ ID NO 30), miR-29b (SEQ ID NO 80), miR-29c (SEQ ID NO 81), is preferred, of which miR-221 (SEQ ID NO 2), miR-222 (SEQ ID NO 3), miR-709 (SEQ ID NO 4), miR-1827 (SEQ ID NO 5) are more preferred, and of which miR-221, optionally in combination with miR-709, is most preferred as an active ingredient in a medicament for the treatment or prevention of liver failure. Using mice as an experimental model representing human patients, it could be shown that the presence of miR-221, transient or permanent, e.g. by transient or permanent over-expression of miR-221, optionally in combination with the presence of miR-709, e.g. by transient or permanent expression, protects hepatocytes in vivo from apoptosis and delays liver failure, especially fulminant liver failure in a patient. For the purposes of the invention, the prevention of liver failure includes the stabilization of a functionally impaired or injured liver, especially of an intoxicated liver, e.g. for administration of the medicament to patients suffering from impaired liver function.

In further experiments it was shown that the inhibition of the mRNA encoding PUMA, which is effectively brought about by the presence of at least one siRNA having a sequence that is reverse complementary to the mRNA encoding PUMA, especially to the 3′UTR of the mRNA encoding PUMA, leads to the reduction or prevention of apoptosis in the presence of an apoptosis inducing agent. Therefore, miR-221 and/or miR-709 are exemplary representatives of those microRNAs which reduce or inhibit the presence or activity of PUMA.

Accordingly, the invention also relates to a nucleic acid construct containing a sequence that encodes at least one of the microRNA molecules hybridizing to the mRNA encoding PUMA, especially to the 3′UTR of the mRNA encoding PUMA, most preferred miR-221 and/or miR-709, as a medicament for the treatment or prevention of liver failure. Such a nucleic acid construct preferably contains a nucleic acid sequence encoding miR-221 functionally linked to an upstream promoter sequence, and preferably to a downstream terminator sequence, e.g. the sequence encoding miR-221 being arranged between an upstream promoter element and a downstream terminator for forming an expression cassette suitable for producing miR-221 by transcription, e.g. within the liver cells of a recipient of the nucleic acid construct.

For permanent expression of the microRNA that reduces or inhibits the presence of PUMA in hepatocytes, the nucleic acid construct containing an expression cassette for this at least one microRNA can have sequences for integration into the genome of hepatocytes, which sequences for integration can e.g. be selected from the group comprising terminal ITR elements, e.g. viral ITR elements, or recombinase recognition elements, especially when the expression cassette is co-transfected or co-transduced with an expression cassette encoding and producing the respective specific recombinase.

### Example 1

### Protection of Hepatocytes Against Induced Apoptosis by miR-221

Primary mouse hepatocytes originating from Balb/C mice, which were purchased from Charles River Laboratories, Germany, were transfected with liposomes containing one microRNA selected from a number of microRNAs which are found both in humans and in mice. In detail, nucleic acid constructs containing an expression cassette containing a coding sequence for the microRNA as given below were formulated as liposomes using liposome complexing reagents. The liposomes containing the nucleic acid constructs having expression cassettes for the microRNAs were transfected to separate cultivated primary mouse hepatocytes at transfection rates of up to 80%. Expression of the microRNAs from the transfected expression cassettes was confirmed by detection of the over-expression of the microRNAs by quantitative reverse transcription PCR (RT-PCR). As a control, the coding sequence for Cre recombinase was included for transcription as a non-specific control RNA. Apoptosis was induced by FAS using the Jo2 antibody at 0.5 μg/mL culture.

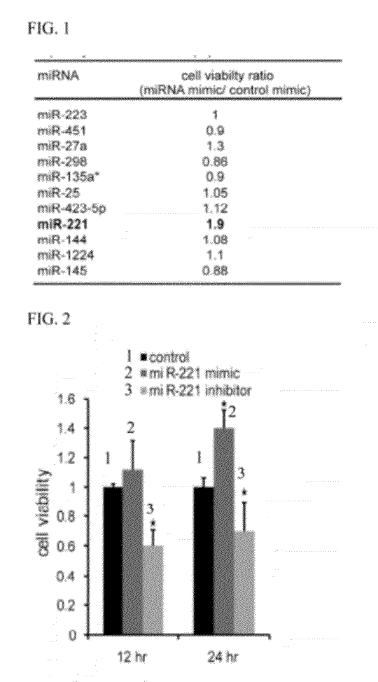

The effect of the expression of the microRNA which was encoded by the transfected nucleic acid construct was assessed by analyzing the hepatocytes. In detail, at 6 hours after seeding, hepatocytes were transfected with the nucleic acid constructs encoding the microRNA, and 12 hours after transfection, apoptosis was induced by supplementing Jo2 antibody to the culture. Apoptosis was detected by exclusion of trypane blue. Hepatocytes were counted at 12 hours after addition of Jo2 antibody. The results show that expression of miR-221 in hepatocytes results in an increased cell viability. FIG. 1 shows the cell viability as the ratio of viable cells for the relevant microRNA in relation to the cell viability obtained for the control micro RNA.

On the example of miR-221, it was shown that inhibition of the mRNA encoding PUMA significantly increased cell viability following induction of apoptosis.

Generally, primarily mouse hepatocytes were isolated by a two-step collagenase (Roche Diagnostics) perfusion followed by a Percoll (obtainable from Sigma) density gradient centrifugation as described by Huh et al, PNAS 4477-4482 (2004). Purified mouse hepatocytes were cultured in Primaria dishes (BD Labware) in hepatocyte basal medium supplemented with hepatocyte single quotes (Lonza). For transfection Targefect hepatocyte reagent and Targefect F2 reagent (both from Targeting Systems) were used according to the manufacturer's instructions.

Analysis of cell viability by WST (obtainable from Roche Diagnostics) analysis shows that miR-221 protects hepatocytes from apoptosis induced by Jo2, whereas an inhibitor of miR-221 enhances the apoptotic effect of Jo2. Results are shown in FIG. 2, wherein results are normalized to the cell viability of controls which were set to 1. Generally, the WST analysis was performed according to the manufacturer's instructions.

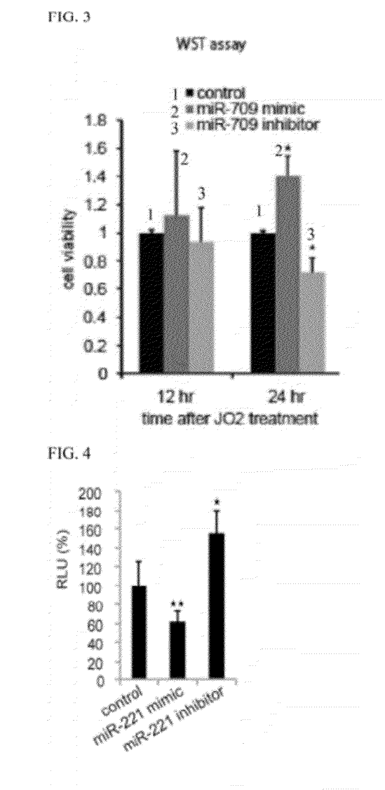

The protective effect of over-expression of the microRNA in hepatocytes from an expression cassette encoding the microRNA that is directed against the mRNA encoding PUMA is also shown on the example of miR-221 analysis of the caspase-3/7 activity. Results are shown in FIG. 3 for miR-221 and in FIG. 4 for miR-709, revealing that hepatocytes transfected with the nucleic acid construct producing miR-221 or miR-709, respectively, have a significantly lower caspase-3/7 activity, whereas hepatocytes which for control were transfected with miR-221 inhibitor or miR-709 inhibitor show an increased activity of caspase-3/7 at 24 hours after induction of apoptosis by treatment with Jo2 antibody.

For further analysis, hepatocytes with a general knockdown of microRNA expression were used. In short, hepatocytes with a knockdown of microRNA production in general were generated by knockdown of DGCR8 in mouse heptoma cells (hepa 1-6, obtained from ATCC), which were cultured in DMEM medium (PAA Laboratories), supplemented with 10% fetal calf serum (PAA), and glutamin (PAA), and penicillin/streptomycin (PAA). The DGCR8 knockdown cells were obtained by transduction with a retroviral vector expressing an shRNA specifically directed against DGCR8 as described by Kumaretal, Nature Genetics 673-677 (2007) and selected in medium supplemented to 1 μg/mL puromycin. Loss of DGCR8 expression was confirmed by Western blotting. Cells were then co-transfected with a nucleic acid construct containing an expression cassette for miR-221.

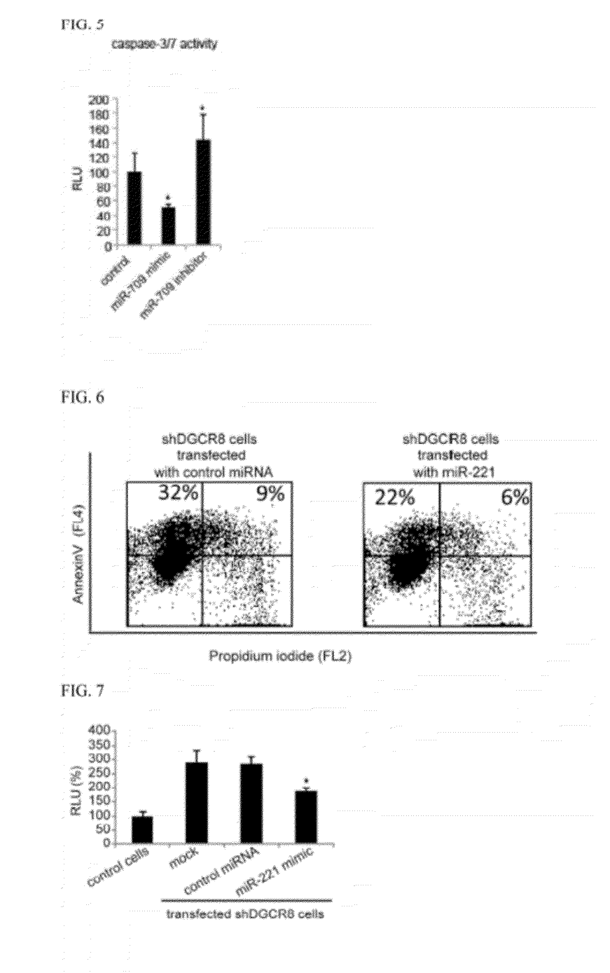

Following induction of apoptosis by addition of Jo2, cells were analyzed by FACS using FL4 for annexin V immune staining. The result is shown in FIG. 6 demonstrating that in contrast to transfectants expressing a control microRNA (control miRNA), transfectants expressing miR-221 (miR-221) show a significantly decreased number of apoptotic cells, supporting the result that it is the presence of a microRNA interfering with the mRNA encoding PUMA which protects hepatocytes from apoptosis, even from fulminant apoptosis as induced by the anti-FAS-antibody Jo2.

The result of the analysis of caspase-3/7 activity in DGCR8-knockdown cells (shDGCR8) is shown in FIG. 7 for non-transfected cells (control), mock-transfected cells (mock), cells containing the expression cassette for a non-specific control microRNA (control miRNA), and for cells containing an expression cassette encoding miR-221 (miR-221 mimic). The caspase-3/7 assay shows a lower caspase-3/7 activity also in the DGCR8 knockdown cells expressing miR-221 in comparison to the mock or control transfected cells.

### Example 2

### MicroRNA Directed Against Puma for Preventing Liver Apoptosis In Vivo

The therapeutic effect of a microRNA which is directed against the mRNA encoding PUMA was shown on mice as experimental animals on FAS-induced fulminant liver failure. For over-expression of the microRNA in vivo, a viral vector containing an expression cassette for the microRNA was used, namely the adeno-associated vector, serotype 8 (AAV8), which has previously been shown by Nakai et al, Journal of Virology 214-224 (2005) to transduce up to 100% of hepatocytes in mice when injected intravenously. Further, Kota et al in Cell 1005-1017 (2009) have shown that the vector AAV8 can successfully be employed for delivery of microRNA to a hepatocellular carcinoma. In short, the viral vector termed AAV8-ttr-miR-221 contained an expression cassette for miR-221, wherein the coding sequence for miR-221 was arranged under the control of the hepatocyte specific transthyritin (Ttr) promoter. The expression cassette was flanked on both sides by inverted terminal repeat elements (ITR) as shown in FIG. 8. Each of the nucleic acid constructs was packaged by viral coat proteins to produce viral particles containing the nucleic acid construct. In short, viral coat proteins were generated as generally described by Gellhaus et al in Hum. Gene Ther. 648-654 (2008). 293T-cells were grown to 50-60% confluency and co-transfected with two plasmids by the calcium precipitation method. One of these plasmids (pDP8.ape, PlasmidFactory, Germany) encodes the viral gene products including rep/cap, E2A, E3, and E4. The Ttr promoter (described by Lu et al, Hum Gene Ther 64-654 (2008)) was ligated in 5′ to the sequence encoding miR-221. At 72 hours after co-transfection of the cultivated cells with pDP8.ape plasmid and the plasmid containing an expression cassette for miR-221 (pD.AAV.Ttr.miR-221) or the control plasmid containing an expression cassette for Crc recombinase (pD.AAV.Ttr.Cre), cells containing viral particles were harvested and AAV8 viral particles were purified according to standard methods. The titer was determined by quantitative RT-PCR using primers spanning the Ttr promoter region (Ttr forward primer SEQ ID NO 85, and Ttr reverse primer SEQ ID NO 86).

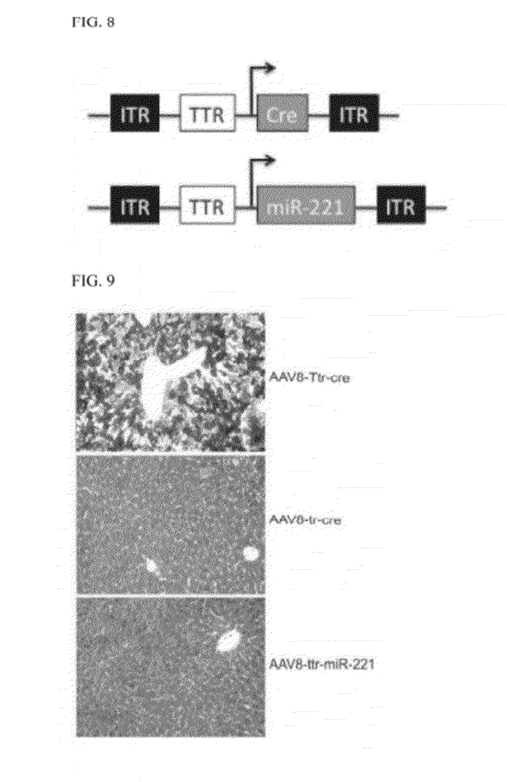

For transduction, 1×10¹¹ viral particles were injected into the tail vein of Balb/C mice. As a control, ROSA26 reporter mice as described by Soriano in Nature Genetics 70-71 (1999) were used for controlling transduction efficiency by injecting 1×10¹¹ viral particles of the control AAV8-Ttr-Cre vector into the tail vein of the ROSA26 Cre reporter mice, which contain a foxed stop codon upstream of the β-galactosidase reporter gene. Efficient hepatocyte transduction could be confirmed by staining for X-Gal of liver sections of ROSA26 mice, as shown in FIG. 9 (AAV8-Ttr-Cre). Further, FIG. 9 shows a micrograph of the liver section of control transduced mice (AAV8-tr-Cre).

FIG. 9 shows a micrograph of a cross-section of a liver of a mouse transfected with viral particles containing the AAV8-Ttr-miR-221 (AAV8-ttr-miR-221) nucleic acid construct, presenting normal histology. Further, normal levels of transaminases were detected in mice having received the viral particles containing the expression cassette for miR-221.

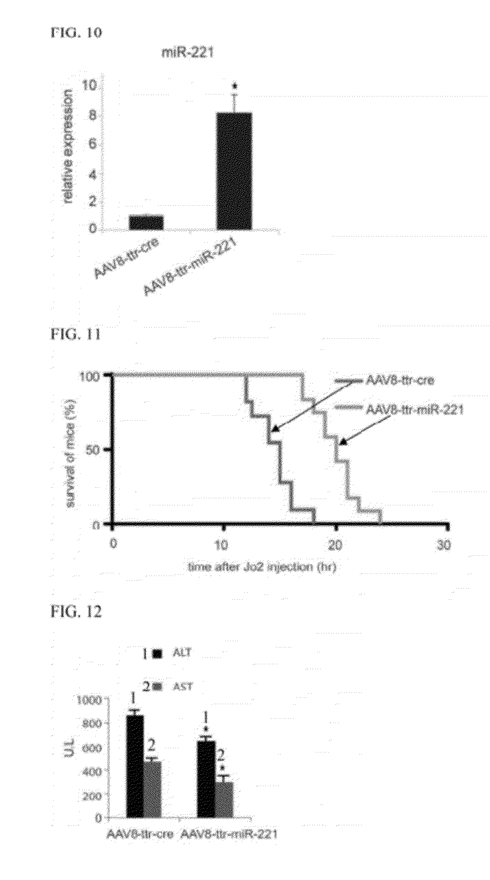

Detection of expression of miR-221 in mice transduced by the viral particles showed an approximately eight-fold higher expression of miR-211 compared to the mice transduced with the control viral particles AAV8-Ttr-Cre four days after injection of the viral particles. FIG. 10 shows a graphic representation of expression levels relative to GAPDH.

For detection of miR-221, total RNA was isolated from purified liver tissue using the miRNeasy kit (Qiagen). Following an on-column DNase treatment, total RNA quality was determined, preferably using the Nanodrop assay (NanoDrop Technologies), and Bioanalyzer (Agilent). For each array, 250 ng RNA was suspended in a specific miRNA hybridization buffer, 25 μL per array), available from FeBIT GmbH, Heidelberg, Germany. Hybridization was performed for 16 hours at 42° C. using the GeniomRT-Analyzer. For quantitative RT-PCR, a real-time RT-PCR reaction was used, with 1 μg RNA for gene expression analysis, and 10 ng total RNA for miRNA expression analysis, respectively, for first strand cDNA synthesis. Preferably, the TaqMan mRNA RT kit was used for cDNA synthesis of miRNAs, and the TaqMan Universal real-time PCR kit was used for quantitative RT-PCR of miRNA, available from Applied Biosystems (USA). For gene expression analysis, e.g. for assaying the mRNA of PUMA, the SYBR Green PCR master mix (Applied Biosystems, USA) was used for real-time PCR. Normalization of gene expression was to expression of GAPDH. Preferably, data were analyzed according to the delta-delta Ct method. For expression analysis of PUMA, forward-primer SEQ ID NO 87 and reverse-primer SEQ ID NO 88 were used, for GAPDH expression analysis, forward-primer of SEQ ID NO 89, and reverse-primer of SEQ ID NO 90 were used.

Consistent over-expression of miR-221 could also be detected in liver tissue up to 2 weeks after in vivo injection of the viral particles containing the nucleic acid construct including the expression cassette for miR-221.

Four days after transduction of mice by injection of the viral particles containing AAV8-Ttr-miR-221 vector for expression of miR-221, and of control-transduced mice, fulminant liver failure was induced by administration of Jo2 antibody intraperitoneally (0.4 μg/g body weight). In the experimental animals, delayed death due to fulminant liver failure was seen in those animals having received the nucleic acid construct for expression of miR-221 compared to control mice; the survival is graphically shown in FIG. 11.

For mice of the control group and of the group over-expressing miR-221 were sacrificed at 9 hours after injection of Jo2 antibody. Pathologic analysis of the livers confirmed that the observed survival effect could be assigned to the presence of miR-221, as these liver tissues showed inhibition of apoptosis, i.e. reduced pathological signs of liver injury. Consistent with liver morphology in the mice over-expressing mirR-221, decreased levels of serum transaminases (ALT and AST) and a reduced apoptosis of hepatocytes by detecting lower caspase-3/7 activity were determined as shown in FIGS. 12 and 13, respectively.

The terminal deoxynucleotidyl transferase-mediated dUTZ nick-end labeling (TUNEL) analysis (available from Millipore, USA) showed a reduced number of TUNEL positive nuclei in the miR-221 expressing mice compared to control mice, as shown in the micrographs of FIG. 14 (magnification 200×, Leica DFC320 microscope). The hematoxylin and eosin (HE) staining confirms reduced apoptosis at 9 h after induction of apoptosis in the livers of mice expressing miR-221 from the expression cassette. These results demonstrate that presence of increased levels of a microRNA inhibiting the mRNA encoding PUMA, e.g. miR-221, in liver tissue delays liver failure by inhibiting the hepatocyte apoptosis when fulminant liver failure is induced by FAS.

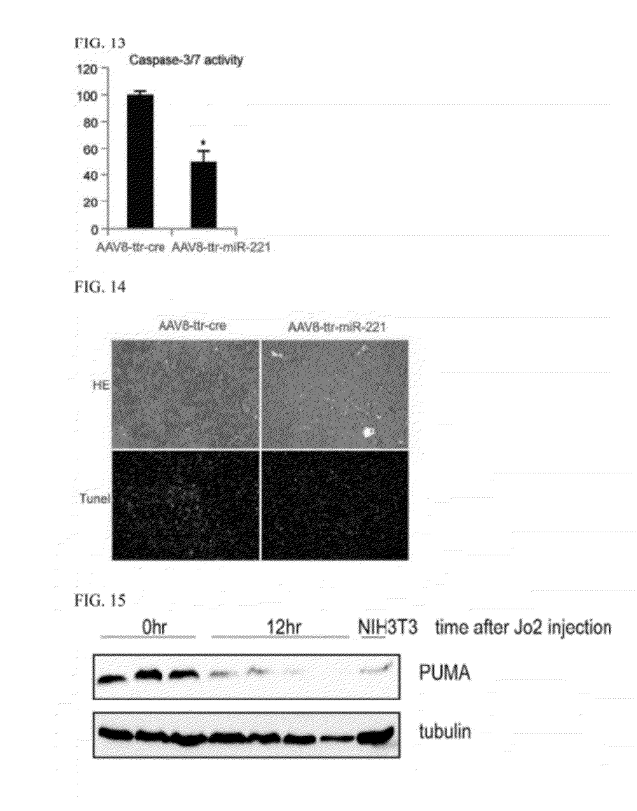

The mechanism of protection of hepatocytes from apoptosis by reducing expression of PUMA was confirmed by analysis of expression of PUMA following induction of apoptosis, i.e. after injection of Jo2. Analysis by Western blotting using an anti-PUMA antibody (1:1000, Abeam), and an anti-tubulin antibody (1:1000, Sigma) show a dramatic reduction of PUMA protein expression at 12 hours after contact with Jo2 antibody, compared to 0 hour control hepatocyte lysates. Transiently elevated levels of PUMA expression at earlier time points did not contradict the reduction of expression of PUMA protein in presence of miR-221 in hepatocytes upon induction of apoptosis. The Western blots are shown in FIG. 15, wherein tubulin serves as an internal control.

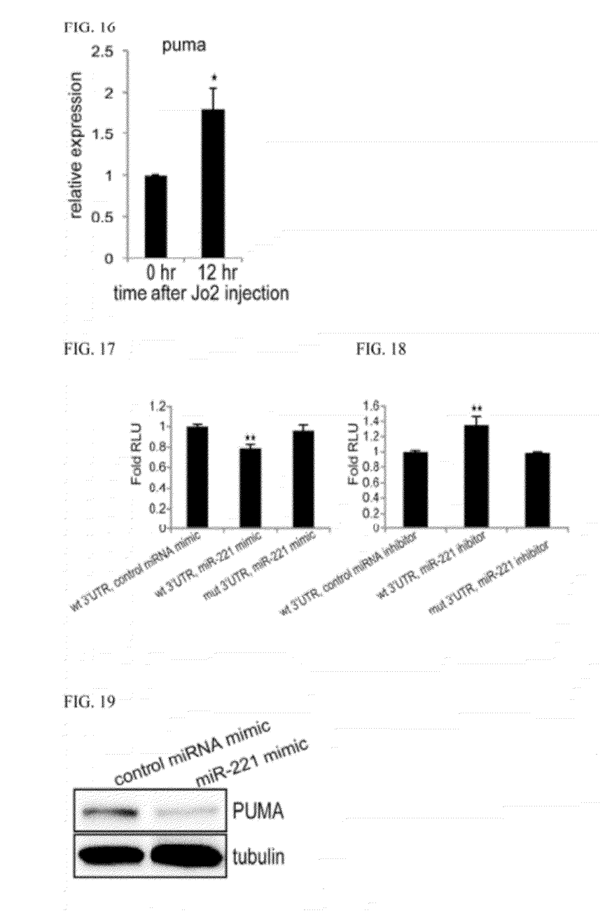

A further analysis of mRNA encoding PUMA by quantitative RT-PCR after induction of apoptosis showed that mRNA levels for PUMA did not decrease, but mRNA encoding PUMA at least in isolated primary hepatocytes increased by a factor of about 1.8 at 12 hours after induction of apoptosis. FIG. 16 shows the results. This observation supports that it is the decrease in protein levels of PUMA which is caused by the microRNA interacting with the mRNA encoding PUMA that protects hepatocytes from apoptosis.

For confirmation, an in vitro assay was used, in which primary hepatocytes were transfected by a nucleic acid construct on the basis of the miR-glo vector, which contains the 3′ UTR of PUMA downstream of the luciferase gene. This luciferase reporter assay using the miR-glo vector demonstrates the direct binding of mature miRNA with the 3′ UTR of the mRNA encoding PUMA. The hepatocytes were transfected to over-express miR-221 and, as a control, expression of miR-221 was inhibited in hepatocytes. The luciferase reporter assay showed that hepatocytes which were transfected with a nucleic acid construct expressing miR-221 (wt 3′UTR, miR-221 mimic) produced lower luciferase activities, whereas hepatocytes which expressed a non-specific miRNA (wt 3′UTR, control miRNA mimic) or inhibitor of miR-221 (wt 3′UTR, miRNA inhibitor) or hepatocytes expressing miR-221 with a mutated 3′UTR (mut 3′UTR, miR-221 mimic) showed a high luciferase activity. This result confirms that miR-221 is specific for reducing expression of Puma through binding to the 3′ UTR of PUMA encoding mRNA. A graphic representation of relative luciferase activities is shown in FIGS. 17 and 18 for hepatocytes transfected with an expression cassette for miR-221, and for hepatocytes transfected with an expression cassette for an inhibitor of miR-221 (miR-221 inhibitor) with wild-type 3′UTR (wt 3′ UTR) or mutated 3′UTR (mut 3′UTR) and non-specific controls (control).

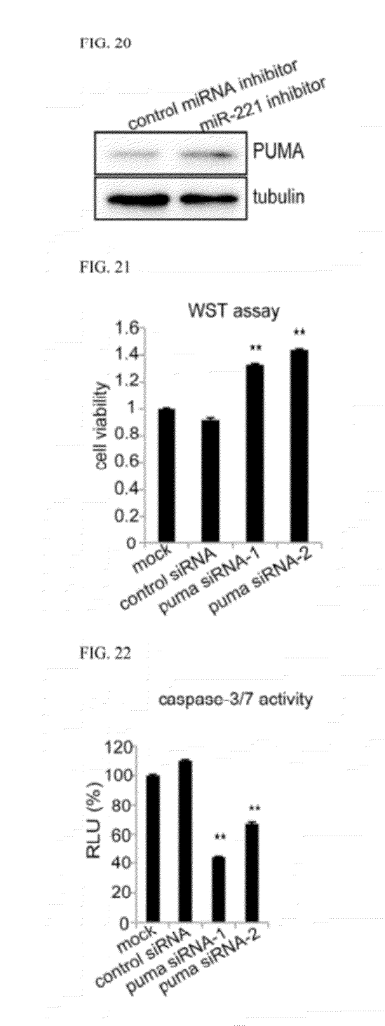

Further, analysis of PUMA protein levels at 48 hours after transfection were decreased in hepatocytes over-expressing miR-221, and increased in hepatocytes in which miR-221 was inhibited, as shown in FIGS. 19 and 20, respectively. Therefore, the above luciferase reporter assay and the finding of altered protein expression after transfection of hepatocytes confirm that it is the reduction of PUMA by targeting of mRNA encoding PUMA through miR-221 over-expression which results in a protection of hepatocytes against apoptosis.

In a further in vitro assay, PUMA expression was knocked-down in primary hepatocytes by miR-221, which is specific for the mRNA encoding PUMA. Analysis of WSC and caspase-3/7 activities showed that loss of PUMA protein protected hepatocytes from apoptosis as induced by Jo2. This result, shown in FIGS. 21 and 22, respectively, was similar to the effect observed after expression of miR-221 in vitro or in vivo.

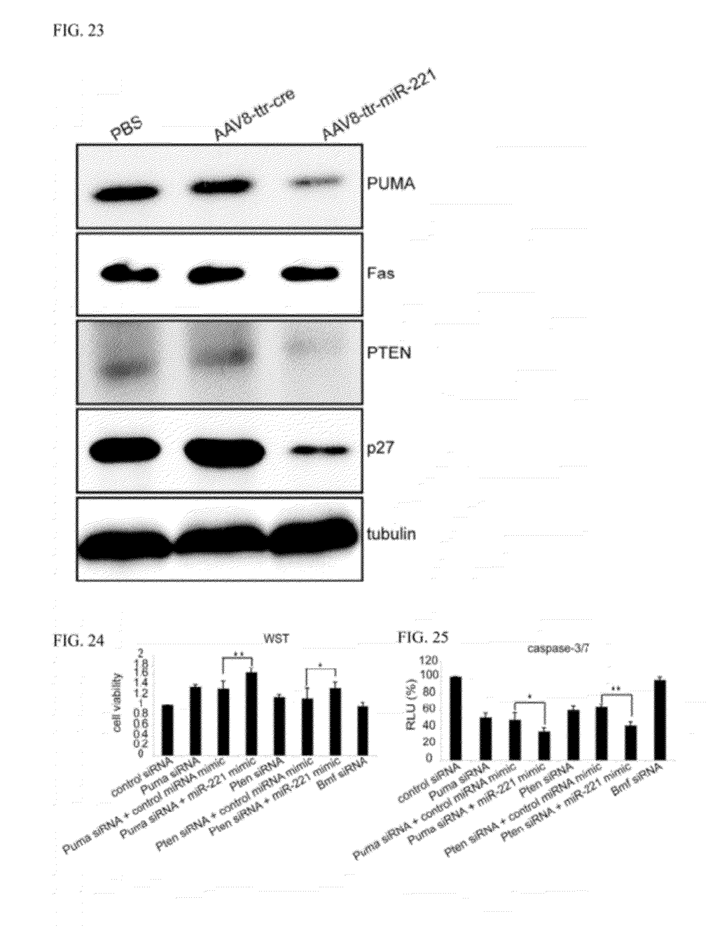

The mice transduced by the viral vector containing the nucleic acid construct AAV8-Ttr-miR-221 were analyzed by Western blotting for PUMA, FAS, p10, p27 and tubulin (loading control) in liver tissue. The result is shown in FIG. 23 showing decreased levels of PUMA protein in the liver tissue of mice over-expressing miR-221, whereas control-transduced mice (AAV8-Ttr-Cre) and mock transduced mice (PBS) showed a similar high expression of PUMA in liver tissue. Further, this analysis shows that expression of FAS receptor was not changed by over-expression of miR-221, suggesting that the anti-apoptotic effect mediated by miR-221 does not involve the FAS receptor.

Further in vitro analysis of hepatocytes in the WST assay and caspase-3/7 activity assay using primary hepatocytes which were knockdown for p10 and BMF using specific siRNA showed that cells transfected with p10 siRNA but not with BMF siRNA were protected against FAS induced apoptosis. Further, transfection with miR-221 in hepatocytes treated with PUMA siRNA or p10 siRNA increased the protection against FAS compared to hepatocytes treated with PUMA siRNA or p10 siRNA alone, as shown in FIGS. 24 and 25. These results demonstrate that PUMA protein and p10 protein show anti-apoptotic behaviour in primary hepatocytes in response to FAS induced apoptosis. Moreover, miR-221 further protects hepatocytes which were treated with siRNA directed against PUMA and p10 from FAS induced apoptosis.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

What is claimed:

1. A medicament for the treatment of a functionally impaired liver, for delaying a deterioration of liver function and/or for prevention of liver failure, containing in a pharmaceutically acceptable formulation an siRNA or microRNA having a nucleic acid sequence which hybridizes to the mRNA encoding PUMA.

2. The medicament of claim 1, wherein the siRNA or microRNA is reverse complementary to 3′ UTR of the mRNA encoding PUMA, which 3′UTR has the sequence of SEQ ID NO: 1.

3. The medicament of claim 1, wherein the siRNA or microRNA comprises a sequence selected from the group consisting of nucleotide sequences having at least 85% sequence similarity to one of SEQ ID NO 2 to SEQ ID NO 84.

4. The medicament of claim 1, wherein the siRNA or microRNA has a sequence selected from the group consisting of nucleotide sequences having at least 85% sequence similarity to one of SEQ ID NO 2 to SEQ ID NO 84, of which the group comprising or consisting of miR-221 (SEQ ID NO 2), miR-222 (SEQ ID NO 3), miR-709 (SEQ ID NO 4), miR-1827 (SEQ ID NO 5), miR-148 (SEQ ID NO 6), miR-152 (SEQ ID NO 7), miR-144 (SEQ ID NO 8), miR-145 (SEQ ID NO 9), miR-139-5p (SEQ ID NO 10), miR-27a (SEQ ID NO 11), miR-27b (SEQ ID NO 78), miR-34a (SEQ ID NO 12), miR-34b-5p (SEQ ID NO 13), miR-34c (SEQ ID NO 14), miR-34c-5p (SEQ ID NO 15), miR-449 (SEQ ID NO 84), miR-449a (SEQ ID NO 16) miR-449b (SEQ ID NO 17), miR-449c (SEQ ID NO 79), miR-699 (SEQ ID NO 18), miR-24 (SEQ ID NO 19), miR-143 (SEQ ID NO 20), miR-101 (SEQ ID NO 21), miR-150 (SEQ ID NO 22), miR-128 (SEQ ID NO 23), miR-125 (SEQ ID NO 24), miR-351 (SEQ ID NO 25), miR-140 (SEQ ID NO 26), miR-140-5p (SEQ ID NO 27), miR-876-3p (SEQ ID NO 28), miR-205 (SEQ ID NO 29), miR-29a (SEQ ID NO 30), miR-29b (SEQ ID NO 80), miR-29c (SEQ ID NO 81).

5. The medicament of claim 1, wherein the siRNA or microRNA is comprised in a nucleic acid construct containing a sequence encoding the siRNA or microRNA in an expression cassette having a promoter that is active in hepatocytes.

6. The medicament of claim 5, wherein the nucleic acid construct is packaged in a viral particle or in a virus-like particle.

7. The medicament of claim 5, wherein the pharmaceutically acceptable formulation contains liposomes containing the siRNA or microRNA.

8. A purified nucleic acid construct containing a sequence encoding an siRNA having a nucleic acid sequence which hybridizes to the mRNA encoding PUMA as a medicament for the treatment of a functionally impaired liver, for delaying a deterioration of liver function and/or for prevention of liver failure.

9. The purified nucleic acid construct according to claim 8, wherein the siRNA has a nucleic acid sequence which is reverse complementary to 3′ UTR of the mRNA encoding PUMA, which 3′UTR has the sequence of SEQ ID NO: 1.

10. The purified nucleic acid construct according to claim 8, wherein the siRNA has a nucleic acid sequence which is selected from the group consisting of nucleotide sequences having at least 85% sequence similarity to one of SEQ ID NO 2 to SEQ ID NO 84.

11. A method for treating, delaying and/or preventing liver failure in humans or animals, the method comprising the step of providing a nucleic acid having which hybridizes to the mRNA encoding PUMA in a pharmaceutical formulation suitable for administration to the human or animal.

12. The method of claim 1 wherein the nucleic acid has a sequence selected from the group consisting of nucleotide sequences having at least 85% sequence similarity to one of SEQ ID NO 2 to SEQ ID NO 84, of which the group comprising or consisting of miR-221 (SEQ ID NO 2), miR-222 (SEQ ID NO 3), miR-709 (SEQ ID NO 4), miR-1827 (SEQ ID NO 5), miR-148 (SEQ ID NO 6), miR-152 (SEQ ID NO 7), miR-144 (SEQ ID NO 8), miR-145 (SEQ ID NO 9), miR-139-5p (SEQ ID NO 10), miR-27a (SEQ ID NO 11), miR-27b (SEQ ID NO 78), miR-34a (SEQ ID NO 12), miR-34b-5p (SEQ ID NO 13), miR-34c (SEQ ID NO 14), miR-34c-5p (SEQ ID NO 15), miR-449 (SEQ ID NO 84), miR-449a (SEQ ID NO 16) miR-449b (SEQ ID NO 17), miR-449c (SEQ ID NO 79), miR-699 (SEQ ID NO 18), miR-24 (SEQ ID NO 19), miR-143 (SEQ ID NO 20), miR-10t (SEQ ID NO 21), miR-150 (SEQ ID NO 22), miR-128 (SEQ ID NO 23), miR-125 (SEQ ID NO 24), miR-351 (SEQ ID NO 25), miR-140 (SEQ ID NO 26), miR-140-5p (SEQ ID NO 27), miR-876-3p (SEQ ID NO 28), miR-205 (SEQ ID NO 29), miR-29a (SEQ ID NO 30), miR-29b (SEQ ID NO 80), miR-29c (SEQ ID NO 81).


SEQUENCE LISTING (90 sequences)

SEQ ID NO: 1 - DNA, 980 bp, Homo sapiens
  3′UTR (1)..(980): 3′UTR of mRNA encoding PUMA
gtgcctgcacccgcccggtggacgtcagggactcggggggcaggcccctcccacctcctgacaccctggccagcgcgggggactttctctgcaccatgtagcatactggactcccagccctgcctgtcccgggggcgggccggggcagccactccagccccagcccagcctggggtgcactgacggagatgcggactcctgggtccctggccaagaagccaggagagggacggctgatggactcagcatcggaaggtggcggtgaccgagggggtggggactgagccgcccgcctctgccgcccaccaccatctcaggaaaggctgttgtgctggtgcccgttccagctgcaggggtgacactgggaggggggggctctcctctcggtgctccttcactctgggcctggcctcaggcccctggtgcttccccccctcctcctgggagggggcccgtgaagagcaaatgagccaaacgtgaccactagcctcctggagccagagagtggggctcgtttgccggttgctccagcccggcgcccagccatcttccctgagccagccggcgggtggtgggcatgcctgcctcaccttcatcagggggtggccaggaggggcccagactgtgaatcctgtgctctgcccgtgaccgccccccgccccatcaatcccattgcataggtttagagagagcacgtgtgaccactggcattcatttggggggtgggagattttggctgaagccgccccagccttagtccccagggccaagcgctggggggaagacggggagtcagggagggggggaaatctcggaagagggaggagtctgggagtggggagggatggcccagcctgtaagatactgtatatgcgctgctgtagataccggaatgaattttctgtacatgtttggttaattttttttgtacatgatttttgtatgtttccttttcaataaaatcagattggaacagtgga

SEQ ID NO: 2 - DNA, 23 bp, Artificial Sequence
  /note=“miR-221, SEQ ID NO 2”
agcuacauugucugcuggguuuc

SEQ ID NO: 3 - RNA, 21 nt, Artificial Sequence
  /note=“miR-222, SEQ ID NO 3”
agcuacaucuggcuacugggu

SEQ ID NO: 4 - RNA, 19 nt, Artificial Sequence
  /note=“miR-709, SEQ ID NO 4”
ggaggcagaggcaggagga

SEQ ID NO: 5 - RNA, 18 nt, Artificial Sequence
  /note=“miR-1827, SEQ ID NO 5”
ugaggcaguagauugaau

SEQ ID NO: 6 - RNA, 22 nt, Artificial Sequence
  /note=“miR-148, SEQ ID NO 6”
ucagugcacuacagaacuuugu

SEQ ID NO: 7 - RNA, 21 nt, Artificial Sequence
  /note=“miR-152, SEQ ID NO 7”
ucagugcaugacagaacuugg

SEQ ID NO: 8 - RNA, 20 nt, Artificial Sequence
  /note=“miR-144, SEQ ID NO 8”
uacaguauagaugauguacu

SEQ ID NO: 9 - RNA, 23 nt, Artificial Sequence
  /note=“miR-145, SEQ ID NO 9”
guccaguuuucccaggaaucccu

SEQ ID NO: 10 - RNA, 22 nt, Artificial Sequence
  /note=“miR-139-5p, SEQ ID NO 10”
ucuacagugcacgugucuccag

SEQ ID NO: 11 - RNA, 21 nt, Artificial Sequence
  /note=“miR-27a, SEQ ID NO 11”
uucacaguggcuaaguuccgc

SEQ ID NO: 12 - RNA, 22 nt, Artificial Sequence
  /note=“miR-34a, SEQ ID NO 12”
uggcagugucuuagcugguugu

SEQ ID NO: 13 - RNA, 23 nt, Artificial Sequence
  /note=“miR-34b-5p, SEQ ID NO 13”
aggcaguguaauuagcugauugu

SEQ ID NO: 14 - RNA, 23 nt, Artificial Sequence
  /note=“miR-34c, SEQ ID NO 14”
aggcaguguaguuagcugauugc

SEQ ID NO: 15 - RNA, 23 nt, Artificial Sequence
  /note=“miR-34c-5p, SEQ ID NO 15”
aggcaguguaguuagcugauugc

SEQ ID NO: 16 - RNA, 22 nt, Artificial Sequence
  /note=“miR-499a, SEQ ID NO 16”
uggcaguguauuguuagcuggu

SEQ ID NO: 17 - RNA, 20 nt, Artificial Sequence
  /note=“miR-499b, SEQ ID NO 17”
aggcaguguuguuagcuggc

SEQ ID NO: 18 - RNA, 20 nt, Artificial Sequence
  /note=“miR-699, SEQ ID NO 18”
aggcagugcgaccuggcucg

SEQ ID NO: 19 - RNA, 22 nt, Artificial Sequence
  /note=“miR-24, SEQ ID NO 19”
uggcucaguucagcaggaacag

SEQ ID NO: 20 - RNA, 21 nt, Artificial Sequence
  /note=“miR-143, SEQ ID NO 20”
ugagaugaagcacuguagcuc

SEQ ID NO: 21 - RNA, 21 nt, Artificial Sequence
  /note=“miR-101, SEQ ID NO 21”
uacaguacugugauaacugaa

SEQ ID NO: 22 - RNA, 22 nt, Artificial Sequence
  /note=“miR-150, SEQ ID NO 22”
ucucccaacccuuguaccagug

SEQ ID NO: 23 - RNA, 21 nt, Artificial Sequence
  /note=“miR-128, SEQ ID NO 23”
ucacagugaaccggucucuuu

SEQ ID NO: 24 - RNA, 24 nt, Artificial Sequence
  /note=“miR-125, SEQ ID NO 24”
ucccugagacccuuuaaccuguga

SEQ ID NO: 25 - RNA, 24 nt, Artificial Sequence
  /note=“miR-351, SEQ ID NO 25”
ucccugaggagcccuuugagccug

SEQ ID NO: 26 - RNA, 22 nt, Artificial Sequence
  /note=“miR-140, SEQ ID NO 26”
cagugguuuuacccuaugguag

SEQ ID NO: 27 - RNA, 22 nt, Artificial Sequence
  /note=“miR-140-5p, SEQ ID NO 27”
cagugguuuuacccuaugguag

SEQ ID NO: 28 - RNA, 22 nt, Artificial Sequence
  /note=“miR-876-3p, SEQ ID NO 28”
uggugguuuacaaaguaauuca

SEQ ID NO: 29 - RNA, 22 nt, Artificial Sequence
  /note=“miR-205, SEQ ID NO 29”
uccuucauuccaccggagucug

SEQ ID NO: 30 - RNA, 22 nt, Artificial Sequence
  /note=“miR-29a, SEQ ID NO 30”
uagcaccaucugaaaucgguua

SEQ ID NO: 31 - RNA, 22 nt, Artificial Sequence
  /note=“miR-494, SEQ ID NO31”
ugaaacauacacgggaaaccuc

SEQ ID NO: 32 - RNA, 23 nt, Artificial Sequence
  /note=“miR-149, SEQ ID NO 32”
ucuggcuccgugucuucacuccc

SEQ ID NO: 33 - RNA, 22 nt, Artificial Sequence
  /note=“miR-874, SEQ ID NO33”
cugcccuggcccgagggaccga

SEQ ID NO: 34 - RNA, 23 nt, Artificial Sequence
  /note=“miR-324-5p, SEQ ID NO 34”
cgcauccccuagggcauuggugu

SEQ ID NO: 35 - RNA, 21 nt, Artificial Sequence
  /note=“miR-326, SEQ ID NO 35”
ccucugggcccuuccuccagu

SEQ ID NO: 36 - RNA, 23 nt, Artificial Sequence
  /note=“miR-330, SEQ ID NO 36”
gcaaagcacacggccugcagaga

SEQ ID NO: 37 - RNA, 22 nt, Artificial Sequence
  /note=“miR-330-5p, SEQ ID NO37”
ucucugggccugugucuuaggc

SEQ ID NO: 38 - RNA, 22 nt, Artificial Sequence
  /note=“miR-136, SEQ ID NO 38”
acuccauuuguuuugaugaugg

SEQ ID NO: 39 - RNA, 21 nt, Artificial Sequence
  /note=“miR-378, SEQ ID NO 39”
acuggacuuggagucagaagg

SEQ ID NO: 40 - RNA, 22 nt, Artificial Sequence
  /note=“miR-422a, SEQ ID NO 40”
acuggacuuagggucagaaggc

SEQ ID NO: 41 - RNA, 22 nt, Artificial Sequence
  /note=“miR-377, SEQ ID NO 41”
aucacacaaaggcaacuuuugu

SEQ ID NO: 42 - RNA, 23 nt, Artificial Sequence
  /note=“miR-325, SEQ ID NO 42”
ccuaguagguguccaguaagugu

SEQ ID NO: 43 - RNA, 22 nt, Artificial Sequence
  /note=“miR-325-3p, SEQ ID NO 43”
uuuauugagcaccuccuaucaa

SEQ ID NO: 44 - RNA, 22 nt, Artificial Sequence
  /note=“miR-532-3p, SEQ ID NO 44”
ccucccacacccaaggcuugca

SEQ ID NO: 45 - RNA, 20 nt, Artificial Sequence
  /note=“miR-665, SEQ ID NO 45”
accaggaggcugaggucccu

SEQ ID NO: 46 - RNA, 22 nt, Artificial Sequence
  /note=“miR-691, SEQ ID NO 46”
auuccugaagagaggcagaaaa

SEQ ID NO: 47 - RNA, 19 nt, Artificial Sequence
  /note=“miR-694, SEQ ID NO 47”
cugaaaauguugccugaag

SEQ ID NO: 48 - RNA, 22 nt, Artificial Sequence
  /note=“miR-763, SEQ ID NO 48”
ccagcugggaagaaccaguggc

SEQ ID NO: 49 - RNA, 18 nt, Artificial Sequence
  /note=“miR-1207-3p, SEQ ID NO 49”
ucagcuggcccucauuuc

SEQ ID NO: 50 - RNA, 21 nt, Artificial Sequence
  /note=“miR-718, SEQ ID NO 50”
cuuccgcccggccgggugucg

SEQ ID NO: 51 - RNA, 22 nt, Artificial Sequence
  /note=“miR-455, SEQ ID NO 51”
uaugugccuuuggacuacaucg

SEQ ID NO: 52 - RNA, 21 nt, Artificial Sequence
  /note=“miR-455-3p, SEQ ID NO 52”
gcaguccaugggcauauacac

SEQ ID NO: 53 - RNA, 22 nt, Artificial Sequence
  /note=“miR-764-3p, SEQ ID NO 53”
aggaggccauaguggcaacugu

SEQ ID NO: 54 - RNA, 22 nt, Artificial Sequence
  /note=“miR-615-5p, SEQ ID NO 54”
ggggguccccggugcucggauc

SEQ ID NO: 55 - RNA, 23 nt, Artificial Sequence
  /note=“miR-423, SEQ ID NO 55”
ugaggggcagagagcgagacuuu

SEQ ID NO: 56 - RNA, 23 nt, Artificial Sequence
  /note=“miR-423-3p, SEQ ID NO 56”
agcucggucugaggccccucagu

SEQ ID NO: 57 - RNA, 23 nt, Artificial Sequence
  /note=“miR-540, SEQ ID NO 57”
caagggucacccucugacucugu

SEQ ID NO: 58 - RNA, 20 nt, Artificial Sequence
  /note=“miR-540-3p, SEQ ID NO 58”
aggucagaggucgauccugg

SEQ ID NO: 59 - RNA, 21 nt, Artificial Sequence
  /note=“miR-703, SEQ ID NO 59”
aaaaccuucagaaggaaagaa

SEQ ID NO: 60 - RNA, 23 nt, Artificial Sequence
  /note=“miR-673, SEQ ID NO 60”
uccggggcugaguucugugcacc

SEQ ID NO: 61 - RNA, 22 nt, Artificial Sequence
  /note=“miR-673-5p, SEQ ID NO 61”
cucacagcucugguccuuggag

SEQ ID NO: 62 - RNA, 22 nt, Artificial Sequence
  /note=“miR-670, SEQ ID NO 62”
aucccugaguguauguggugaa

SEQ ID NO: 63 - RNA, 22 nt, Artificial Sequence
  /note=“miR-711, SEQ ID NO 63”
gggacccggggagagauguaag

SEQ ID NO: 64 - RNA, 22 nt, Artificial Sequence
  /note=“miR-695, SEQ ID NO 64”
agauugggcauaggugacugaa

SEQ ID NO: 65 - RNA, 22 nt, Artificial Sequence
  /note=“miR-331, SEQ ID NO 65”
cuagguauggucccagggaucc

SEQ ID NO: 66 - RNA, 21 nt, Artificial Sequence
  /note=“miR-712, SEQ ID NO 66”
gccccugggccuauccuagaa

SEQ ID NO: 67 - RNA, 22 nt, Artificial Sequence
  /note=“miR-300, SEQ ID NO 67”
uaugcaagggcaagcucucuuc

SEQ ID NO: 68 - RNA, 20 nt, Artificial Sequence
  /note=“miR-343.m, SEQ ID NO 68”
ucucccuccgugugcccaga

SEQ ID NO: 69 - RNA, 22 nt, Artificial Sequence
  /note=“miR-323-5p, SEQ ID NO 69”
aggugguccguggcgcguucgc

SEQ ID NO: 70 - RNA, 22 nt, Artificial Sequence
  /note=“miR-342-5p, SEQ ID NO 70”
aggggugcuaucugugauugag

SEQ ID NO: 71 - RNA, 21 nt, Artificial Sequence
  /note=“miR-188, SEQ ID NO 71”
cucccacaugcaggguuugca

SEQ ID NO: 72 - RNA, 21 nt, Artificial Sequence
  /note=“miR-188-5p, SEQ ID NO 72”
caucccuugcaugguggaggg

SEQ ID NO: 73 - RNA, 22 nt, Artificial Sequence
  /note=“miR-710, SEQ ID NO 73”
ccaagucuuggggagaguugag

SEQ ID NO: 74 - RNA, 21 nt, Artificial Sequence
  /note=“miR-680, SEQ ID NO 74”
gggcaucugcugacauggggg

SEQ ID NO: 75 - RNA, 23 nt, Artificial Sequence
  /note=“miR-298.mr, SEQ ID NO 75”
ggcagaggagggcuguucuuccc

SEQ ID NO: 76 - RNA, 23 nt, Artificial Sequence
  /note=“miR-493, SEQ ID NO 76”
ugaagguccuacugugugccagg

SEQ ID NO: 77 - RNA, 22 nt, Artificial Sequence
  /note=“miR-690, SEQ ID NO 77”
aaaggcuaggcucacaaccaaa

SEQ ID NO: 78 - RNA, 21 nt, Artificial Sequence
  /note=“miR-27b, SEQ ID NO 78”
uucacaguggcuaaguucugc

SEQ ID NO: 79 - RNA, 21 nt, Artificial Sequence
  /note=“miR-449c, SEQ ID NO 79”
aggcagugcauugcuagcugg

SEQ ID NO: 80 - RNA, 23 nt, Artificial Sequence
  /note=“miR-29b, SEQ ID NO 80”
uagcaccauuugaaaucaguguu

SEQ ID NO: 81 - RNA, 22 nt, Artificial Sequence
  /note=“miR-29c, SEQ ID NO 81”
uagcaccauuugaaaucgguua

SEQ ID NO: 82 - RNA, 21 nt, Artificial Sequence
  /note=“miR-331-3p, SEQ ID NO 82”
gccccugggccuauccuagaa

SEQ ID NO: 83 - RNA, 22 nt, Artificial Sequence
  /note=“miR-300-3p, SEQ ID NO 83”
uaugcaagggcaagcucucuuc

SEQ ID NO: 84 - RNA, 21 nt, Artificial Sequence
  /note=“miR-449, SEQ ID NO 84”
uuaagacuugcagugauguuu

SEQ ID NO: 85 - DNA, 19 bp, Artificial Sequence
  /note=“Ttr forward primer, SEQ ID NO 85”
tcagcttggcagggatcag

SEQ ID NO: 86 - DNA, 20 bp, Artificial Sequence
  /note=“Ttr backward primer, SEQ ID NO 86”
gacggcttctcctggtgaag

SEQ ID NO: 87 - DNA, 20 bp, Artificial Sequence
  /note=“PUMA forward primer, SEQ ID NO 87”
ctgtatcctgcagcctttgc

SEQ ID NO: 88 - DNA, 19 bp, Artificial Sequence
  /note=“PUMA backward primer, SEQ ID NO 88”
acgggcgactctaagtgct

SEQ ID NO: 89 - DNA, 18 bp, Artificial Sequence
  /note=“GAPDH forward primer, SEQ ID NO 89”
atggccttccgtgttcct

SEQ ID NO: 90 - DNA, 16 bp, Artificial Sequence
  /note=“GAPDH backward primer, SEQ ID NO 90”
cggcacgtcagatcca